(19) United States
(12) Patent Application Publication
(10) Pub. No.: US 2013/0168227 A1
(43) Pub. Date: Jul. 4, 2013

(54) PROCESS FOR THE PRODUCTION OF FURFURAL

(71) Applicant: E I DU PONT DE NEMOURS AND COMPANY, Wilmington, DE (US)

(72) Inventors: Paul Joseph Fagan, Wilmington, DE (US); Ronnie Ozer, Arden, DE (US); Eric J. Till, Newtown Square, PA (US)

(73) Assignee: E I DU PONT DE NEMOURS AND COMPANY, Wilmington, DE (US)

(21) Appl. No.: 13/729,526

(22) Filed: Dec. 28, 2012

Related U.S. Application Data
(60) Provisional application No. 61/580,728, filed on Dec. 28, 2011.
(60) Provisional application No. 61/580,733, filed on Dec. 28, 2011.

(51) Int. Cl. C07D 307/48

(52) U.S. Cl. CPC C07D 307/48
USPC 203/35; 203/34

(57) ABSTRACT

Furfural is produced in one step at both high yield and high conversion from a feedstock comprising solid biomass and/or insoluble polysaccharide, in a high boiling, water-miscible solvent containing a soluble acid catalyst, and water. Furfural product and water can be distilled off, leaving non-volatile solvent behind. Because furfural contact with the acidic medium is minimized, degradation is kept to a minimum. The feedstock does not have to be pretreated. Because the biomass undergoes near complete dissolution, the residual material is flowable and easier to handle than residual solids reported from other processes. Further, certain by-products (e.g., humins, lignins) solubilized in the reaction solvent can be precipitated by addition of water or aqueous solution and then removed from the reaction mixture.

This application claims priority under 35 U.S.C. §119(e) from, and claims the benefit of, U.S. Provisional Application No. 61/580,728, filed Dec. 28, 2011; and U.S. Provisional Application No. 61/580,733, filed Dec. 28, 2011; each of which is by this reference incorporated in its entirety as a part hereof for all purposes.

### FIELD OF THE INVENTION

A method for the production of furfural and other chemicals from solid biomass is provided.

### BACKGROUND

Furfural and related compounds are useful precursors and starting materials for industrial chemicals for use as pharmaceuticals, herbicides, stabilizers, and polymers. The current furfural manufacturing process utilizes biomass such as corn cob or sugar cane bagasse as a raw material feedstock for obtaining glucose, glucose oligomers, cellulose, xylose, xylose oligomers, arabinose, hemicellulose, and other C₅ and C₆ sugar monomers, dimers, oligomers, and polymers. The hemicellulose and cellulose are hydrolyzed under acidic conditions to their constituent sugars, such as glucose, xylose, mannose, galactose, rhamnose, and arabinose. Xylose, which is a pentose (i.e., a C₅ monosaccharide) is the sugar present in the largest amount in hemicellulose. In a similar aqueous acidic environment, the C₅ sugars are subsequently dehydrated and cyclized to furfural. Under similar conditions, C₆ sugars can be hydrolyzed and converted in low yields to furfural.

Alternatively, furfural can be produced directly from the solid biomass in a single step. In the latter scenario, the cellulose and lignin fractions of the biomass can be further utilized for their burn value, for other products or even as an additional source of furfural. Several methods include attempting to dissolve, partially dissolve or dissolve and chemically react the biomass with the aid of an organic solvent. For example, L. D. Starr et al. delignified hemlock and pine at 160° C.-190° C. using water and sulfolane mixtures (Tappi Alkaline Pulping Conference Preprints. 1975, 195-198). L. P. Clermont delignified aspen using water and sulfolane under varying conditions (Tappi, 1970, 53, 2243-2245). Saka et al. (J. Wood. Sci. 2007, 53, 127-133) disclose a reaction in which cellulose is pyrolyzed in sulfolane at 200° C.-280° C. with an acid catalyst to produce furfural and other compounds.

In a process disclosed by Gregory R. Court et al. (PCT International Application WO 2011/000030(A1)) lignocellulose (biomass) was heated with an organic solvent and acid catalyst to form a char, releasing 10-40% volatile organic compounds of which <10% is furfural.

However, producing furfural directly from solid biomass in high yield has been difficult and materials of construction can be expensive for aqueous based processes done at higher pressures. There remains a need for a process to produce furfural from solid biomass in an efficient and cost-effective manner.

### SUMMARY OF THE INVENTION

In an aspect of the invention, there is a process comprising:
(a) providing a reactor comprising a distillation column disposed on top of a reaction vessel, wherein the reaction vessel contains a biomass feedstock, water, a soluble acid catalyst and a water-miscible organic solvent having boiling point of at least 100° C., wherein the biomass feedstock comprises solid biomass, insoluble polysaccharide or a mixture thereof;
(b) adding water to the reaction vessel and bringing the contents of the reaction vessel to a temperature in the range of 100-250° C. and a pressure in the range of 0.0001-0.21 MPa to form a reaction mixture for a residence time sufficient to produce a mixture of water and furfural; and
(c) removing the mixture of water and furfural from the top of the distillation column.

In an embodiment, the process is a continuous process further comprising:
(d) distilling at least a portion of the contents of the reaction vessel to remove distillates including levulinic acid, that have boiling points between that of furfural/water mixture and the water-miscible organic solvent;
(e) removing solid materials from the remaining mixture of step (d) by filtration or centrifugation;
(f) adding water to the remaining mixture of step (d) and precipitating humins, lignin, and insoluble byproducts;
(g) separating the liquid from the solids produced in step (f); and
(h) adding at least one of soluble acid catalyst or water-miscible organic solvent to the solid-free liquid obtained in step (g) and adding it to the reaction vessel as in step (a).

In another aspect there is a process comprising the steps of:
a) providing a water-miscible organic solvent and a soluble acid catalyst in a reaction vessel, wherein the boiling point of the solvent is higher than about 100° C.,
b) adding feedstock and, optionally, water in the form of liquid and/or steam to the reaction vessel to form a reaction mixture wherein
   i) the feedstock comprises solid biomass and/or insoluble polysaccharide,
   ii) the temperature of the reaction mixture is between about 100° C. and about 250° C.,
   iii) the reaction mixture pressure is between 0 MPa and about 0.21 MPa, and
   iv) the feedstock, organic solvent, and catalyst are in contact for a time sufficient to effect a reaction to produce furfural and water; and
c) removing vapors of furfural and water from the reaction mixture via reflux through a multistage distillation column;
d) condensing and collecting a solution comprising furfural and water; and
e) recovering furfural from the mixture removed in step d).

In a further embodiment of the invention disclosed herein, a process is provided comprising the steps of:
a) providing a water-miscible organic solvent and a soluble acid catalyst in a reaction vessel, wherein the boiling point of the solvent is higher than about 100° C.;
b) heating the contents of the reaction vessel to a temperature between about 100 and about 250° C.;
c) adding feedstock and, optionally, water in the form of liquid and/or steam to the reaction vessel to form a reaction mixture wherein
   i) the feedstock comprises solid biomass and/or insoluble polysaccharide,
   ii) the feedstock is present at about 1 to about 90 weight percent based on the weight of the reaction mixture,
   iii) the reaction mixture pressure is between 0 MPa and about 0.21 MPa, and
   iv) the feedstock, organic solvent, and catalyst are in contact for a time sufficient to effect a reaction to produce furfural and water;
d) removing vapors of furfural and water from the reaction mixture via reflux through a multistage distillation column;
e) condensing and collecting a solution comprising furfural and water;
f) feeding the remaining contents of the reaction vessel, or a portion thereof, through a filter or screen to a high boiler distillation vessel wherein higher boilers, including levulinic acid, are distilled at a pressure of 0 MPa to 0.21 MPa;
g) optionally, removing remaining solids from the reaction vessel for recycle into the process, or for removal from the process;
h) removing solids from the high boiler distillation vessel after the removal of high boilers in step f);
i) feeding remaining solution from the high boiler distillation vessel, or a portion thereof, to a mixing chamber and diluting the solution with water thereby precipitating water-insoluble material;
j) removing the water-insoluble material precipitated in step i); and
k) feeding the solution remaining after step j) back to the reaction vessel.

### BRIEF DESCRIPTION OF THE DRAWINGS

Various features and/or embodiments of this invention are illustrated in drawings as described below. These features and/or embodiments are representative only, and the selection of these features and/or embodiments for inclusion in the drawings should not be interpreted as an indication that subject matter not included in the drawings is not suitable for practicing the invention, or that subject matter not included in the drawings is excluded from the scope of the appended claims and equivalents thereof.

FIG. 1 is a schematic illustration of an exemplary reactor configuration used in the production of furfural in a batch mode, in accordance with various embodiments of the present invention.

FIG. 2 is a schematic illustration of an exemplary reactor configuration used in the production of furfural in a continuous mode, in accordance with various embodiments of the present invention.

### DETAILED DESCRIPTION

### Definitions

As used herein, the term “sugar” includes monosaccharides, disaccharides, and oligosaccharides. Monosaccharides, or “simple sugars,” are aldehyde or ketone derivatives of straight-chain polyhydroxy alcohols containing at least three carbon atoms. A pentose is a monosaccharide having five carbon atoms; some examples are xylose, arabinose, lyxose and ribose. A hexose is a monosaccharide having six carbon atoms; some examples are glucose and fructose. Disaccharide molecules (e.g., sucrose, lactose, and maltose) consist of two covalently linked monosaccharide units. As used herein, “oligosaccharide” molecules consist of about 3 to about 20 covalently linked monosaccharide units.

As used herein, the term “polysaccharide” means a polymer consisting of over 20 covalently linked monosaccharide units. Polysaccharides may be linear or branched. Some examples are cellulose, starch, and glycogen.

As used herein, the term “Cₙ sugar” includes monosaccharides having n carbon atoms; disaccharides comprising monosaccharide units having n carbon atoms, and oligosaccharides comprising monosaccharide units having n carbon atoms. Thus, “C₅ sugar” includes pentoses, disaccharides comprising pentose units, and oligosaccharides comprising pentose units.

As used herein, the term “hemicellulose” refers to a polymer comprising C₅ and C₆ monosaccharide units. Hemicellulose consists of short, highly branched chains of sugars. In contrast to cellulose, which is a polymer of only glucose, a hemicellulose is a polymer of five different sugars. It contains five-carbon sugars (usually D-xylose and L-arabinose) and six-carbon sugars (D-galactose, D-glucose, and D-mannose). Hemicellulose can also contain uronic acid, sugars in which the terminal carbon's hydroxyl group has been oxidized to a carboxylic acid, such as, D-glucuronic acid, 4-O-methyl-D-glucuronic acid, and D-galacturonic acid. The sugars are partially acetylated. Typically the acetyl content is 2 to 3% by weight of the total weight of the hemicellulose. Xylose is typically the sugar monomer present in hemicellulose in the largest amount.

As used herein, the term “biomass” refers to organic materials that are plant or animal based, including but not limited to dedicated energy crops, agricultural crops and trees, food, feed and fiber crop residues, aquatic plants, forestry and wood residues, agricultural wastes, biobased segments of industrial and municipal wastes, processing by-products and other non-fossil organic materials. Three main categories of biomass are primary, secondary and tertiary biomass Primary biomass is biomass produced directly by photosynthesis and harvested or collected from the field or forest where it is grown. Examples are grains, perennial grasses and wood crops, crop residues such as sugar cane bagasse, corn stover, corn cobs, and residues from logging and forest operations. Secondary biomass includes residues and byproduct streams from food, feed, fiber, wood and materials processing plants (such as sawdust, black liquor, cheese whey, biomass prehydrolysate from pulp and paper mills), and manures from concentrated animal feeding operations. Tertiary biomass includes post consumer residues and wastes, such as construction and demolition wood debris, other waste wood from urban environments, as well as packaging wastes, and municipal solid wastes.

As used herein, the term “high boiling” as applied to a solvent denotes a solvent having a boiling point above about 100° C. at one atmosphere.

As used herein the term “water-miscible organic solvent” refers to an organic solvent that can form a monophasic solution with water at the temperature at which the reaction is carried out.

As used herein, the term “higher boiler” denotes a reaction product or byproduct that has a boiling point between that of the furfural/water azeotrope and that of the high boiling solvent. An example of a higher boiler when sulfolane is the high boiling solvent is levulinic acid.

As used herein, the term “organic” denotes carbon-containing compounds with the following exceptions: binary compounds as the carbon oxides, carbides, carbon disulfide, etc.; ternary compounds such as metallic cyanides, metallic carbonyls, phosgene, carbonylsulfide; and metallic carbonates such as calcium carbonate and sodium carbonate.

As used herein, the term “catalytic amount” means a substoichiometric amount of catalyst relative to a reactant.

As used herein, the term “organic acid” means an organic compound having acidic properties; some examples are acetic acid, formic acid, and methane sulfonic acid.

As used herein, the term “mineral acid” means an inorganic acid, as distinguished from organic acid. Some examples are sulfuric acid, nitric acid, phosphoric acid, and hydrochloric acid.

As used herein, the term “heteropolyacid” denotes an oxygen-containing acid with P, As, Si, or B as a central atom which is connected via oxygen bridges to W, Mo or V. Some examples are phosphotungstic acid, molybdophosphoric acid.

As used herein the term “humin(s)” refers to dark, amorphous byproduct(s) resulting from acid induced sugar and furfural degradation.

As used herein, the term “selectivity” refers to the moles of furfural produced, divided by the moles of xylose transformed to products over a particular time period.

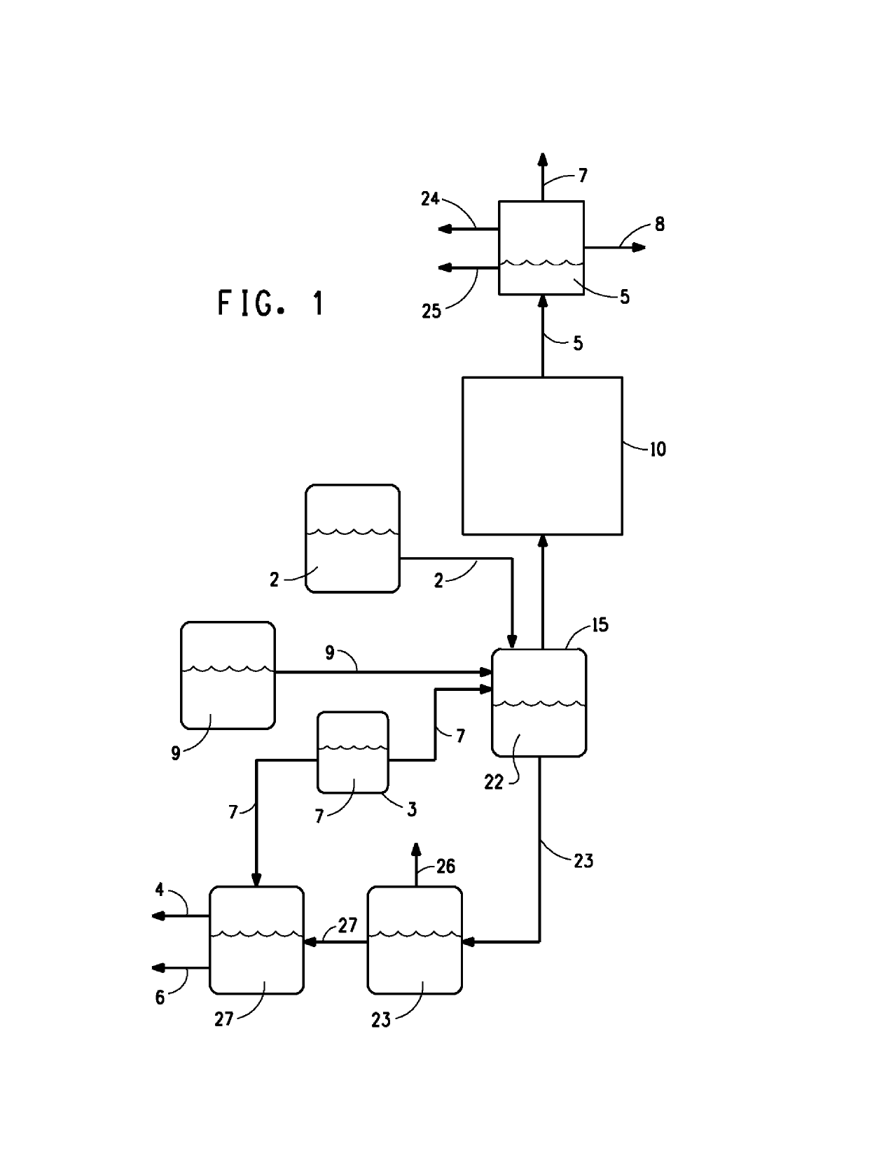

In an embodiment, there is a process for the production of furfural comprising providing a reactor configuration comprising a distillation column disposed on top of a reaction vessel, wherein the reaction vessel contains a biomass feedstock, water, a soluble acid catalyst and a water-miscible organic solvent. FIG. 1 shows a schematic illustration of an exemplary reactor configuration comprising a distillation column 10 disposed on top of a reaction vessel 15, wherein the reaction vessel 15 contains a reaction mixture 22 comprising a biomass feedstock, water, a soluble acid catalyst and a water-miscible organic solvent. In another embodiment, the solid biomass can be added to the solution of soluble acid catalyst and water-miscible solvent in the reaction vessel 15 over a period of time to form reaction mixture 22.

The water-miscible organic solvent has a boiling point higher than about 100° C. at atmosphere pressure. Examples of suitable water-miscible organic solvents include without limitation: sulfolane, polyethylene glycol, isosorbide dimethyl ether, isosorbide, propylene carbonate, poly(ethylene glycol) dimethyl ether, adipic acid, diethylene glycol, 1,3-propanediol, glycerol, gamma-butyrolactone, 1-methyl-2-pyrrolidinone, and gamma-valerolactone. In one embodiment, the polar, water-miscible organic solvent is sulfolane. In an embodiment, the water-miscible organic solvent is PEG 4600, PEG 10000, PEG 1000, gamma-valerolactone, gamma-butyrolactone, isosorbide dimethyl ether, propylene carbonate, adipic acid, poly(ethylene glycol)dimethyl ether, isosorbide, Cerenol™ 270 (poly(1,3-propanediol), Cerenol™ 1000 ((poly(1,3-propanediol)), or diethylene glycol.

The soluble acid catalyst is water-soluble and comprises a mineral acid, a heteropolyacid, an organic acid, or a combination thereof. In one embodiment, the acid catalyst is a mineral acid comprising sulfuric acid, phosphoric acid, hydrochloric acid, or a combination of these. In another embodiment, the acid catalyst is a heteropolyacid comprising phosphotungstic acid, molybdophosphoric acid, or a combination of these. In other embodiment, the acid catalyst is an organic acid comprising oxalic acid, formic acid, acetic acid, an alkyl sulfonic acid, an aryl sulfonic acid, a halogenated acetic acid, a halogenated alkylsulfonic acid, a halogenated aryl sulfonic acid, or a combination of these. An example of a suitable alkyl sulfonic acid is methane sulfonic acid. An example of a suitable aryl sulfonic acid is toluenesulfonic acid. An example of a suitable halogenated acetic acid is trichloroacetic acid. An example of a suitable halogenated alkylsulfonic acid is 1,1,2,2-tetrafluoroethanesulfonic acid. An example of a suitable halogenated aryl sulfonic acid is fluorobenzenesulfonic acid.

The soluble acid catalyst is present in the water-miscible organic solvent in the range of 0.01-11 weight % or 0.01-5 weight % or 0.1-1.5 weight %, based on the total weight of the acid solution (solvent and acid). In some embodiments, the acid is present in the solvent at a weight percentage between and optionally including any two of the following values: 0.01, 0.05, 0.10, 0.15, 0.20, 0.50, 1.0, 1.5, 2.0, 3.0, 4.0, 5.0, 6.0, 7.0, 8.0, 9.0, and 10 weight percent. The optimal amount of acid catalyst will be affected by what specific solvent is used and is readily determined by one of skill in the art.

The process for the production of furfural also comprises, as shown in the FIG. 1, adding water 7 from vessel 3 to the reaction vessel and bringing the contents of the reaction vessel to a temperature in the range of 100-250° C. and a pressure in the range of 0.0001-0.21 MPa to form a reaction mixture 22 in the reaction vessel 15 for a residence time sufficient to produce a mixture 5 of water 7 and furfural 8. In an embodiment, feedstock comprises solid biomass, insoluble polysaccharide or a mixture thereof.

The source of the biomass feedstock is not determinative of the invention, and the biomass may be from any source, and can include purified insoluble forms of biomass such as cellulose or other polysaccharides. Biomass can be derived from a single source, or biomass can comprise a mixture derived from more than one source; for example, biomass could comprise a mixture of corn cobs and corn stover, or a mixture of grass and leaves. Biomass includes, but is not limited to, bioenergy crops, agricultural residues, municipal solid waste, industrial solid waste, sludge or waste streams from paper or pulp manufacture, yard waste, wood and forestry waste or a combination thereof. Examples of biomass include, but are not limited to, corn grain, corn cobs, crop residues such as corn husks, corn stover, grasses, wheat, wheat straw, barley, barley straw, hay, rice straw, switchgrass, waste paper, sugar cane bagasse, sorghum, soy, components obtained from milling of grains, trees, branches, roots, leaves, wood chips, sawdust, shrubs and bushes, vegetables, fruits, flowers, and animal manure or a combination thereof. Suitable biomass for use in the processes disclosed herein can include biomass that has a relatively high carbohydrate value, is relatively dense, and/or is relatively easy to collect, transport, store and/or handle. In one embodiment of the invention, biomass that is useful includes corn cobs, wheat straw, chipped wood, sawdust, and sugar cane bagasse.

The biomass feedstock may be used directly as obtained from the source, or energy may be applied to the biomass to reduce the size, increase the exposed surface area, and/or increase the availability of lignin, cellulose, hemicellulose, and/or oligosaccharides present in the biomass to the acid catalyst, to the water, and to the high boiling organic solvent. Energy means useful for reducing the size, increasing the exposed surface area, and/or increasing the availability of lignin, cellulose, hemicellulose, and/or oligosaccharides present in the biomass feedstock include, but are not limited to, milling, crushing, grinding, shredding, chopping, disc refining, ultrasound, and microwave. This application of energy may occur before and/or during contacting with the water and/or immiscible organic solvent. The biomass feedstock may be used directly as obtained from the source or may be dried to reduce the amount of moisture contained therein.

In some embodiments, the feedstock can be contacted with the water-miscible solvent and acid catalyst as dry solids, as damp solids, or in a slurry (e.g., of water or solvent) with loadings of from 1 to 90 weight % or 10-85 weight % or 20-80 weight %. In some embodiments, the feedstock loading is between and optionally including any two of the following values: 5, 10, 20, 30, 40, 50, 60, 70, 80, and 90 weight %.

The temperature of the reaction mixture 22 in the reaction vessel 15 is between about 100-250° C. or 100-190° C. or 120-180° C. In some embodiments, the temperature of the reaction mixture is between and optionally including any two of the following values: 100, 110, 120, 130, 140, 150, 160, 170, 180, 190, 200, 210, 220, 230, 240, and 250° C.

The reaction is carried at a pressure less than about 0.21 Mpa, thus eliminating the need for the high-pressure equipment. In an embodiment, the reaction mixture 22 is kept at a pressure less than 0.21 Mpa or less than 0.11 MPa or less than 0.050 MPa.

The process for the production of furfural further comprises, as shown in the FIG. 1, removing the mixture 5 of water 7 and furfural 8 from the top of the distillation column 10. As the reaction proceeds, vapors of furfural 8 and water 7 are removed from the reaction mixture 22 via reflux through a multistage distillation column 10, condensed, and collected as a solution 5 of furfural 8 and water 7. The use of staging in the distillation process allows more efficient stripping of furfural away from reaction zone 15, and minimizes loss of water miscible solvent overhead. This increases furfural yield by driving the reaction toward completion and by minimizing formation of byproducts.

In one embodiment, a batch mode, with reference to FIG. 1, the reaction vessel 15 is charged initially with a water-miscible solvent, feedstock (biomass and/or insoluble polysaccharide), water and a catalytic amount of soluble acid. The contents of the reaction vessel 15 are heated to the reaction temperature. In another embodiment, the feedstock (biomass and/or insoluble polysaccharide) is added to the hot reaction vessel 15 over time. A stream of water 7 from vessel 3 and/or steam is optionally also added continuously to the reaction vessel 15. Sugars are produced by acid hydrolysis of the feedstock and then undergo chemical transformation to furfural 8, which is then distilled from the reaction mixture 22 along with water 7 produced by the reaction and from any water or steam added to the reaction vessel 15. This minimizes the residence time of furfural 8 in the acidic environment of the reaction mixture 22 and thereby minimizes its degradation. Other useful products that may distill with furfural and water are formic acid 24 and acetic acid 25. The furfural, as well as any other desired products that may be present, are separated from the water and purified by any convenient methods known in the art, and the product furfural 8 is removed. The water 7 is either recycled to the water stream feed, the biomass feed, or is released from the process. After the completion of the reaction, non-volatile components are left in the reaction vessel 15 including, but not limited to, the high boiling, water-miscible solvent, soluble acid, unreacted feedstock, and unwanted water-insoluble byproducts such as humins. The reaction solution 23 remaining in the reaction vessel 15 can be removed through a filter or screen to separate 23 from insoluble fines (eg. feedstock residue). Any solid feedstock residue can be removed and recycled into the process, or can be released from the process. Optionally, other products can be distilled from reaction solution 23 such as levulinic acid 26 at atmospheric or low pressure (0.0001-0.11 MPa). The water-insoluble byproducts in the reaction solution can then be removed by diluting at least a portion of 23 with water 7 from vessel 3, which results in the precipitation of humins, followed by, e.g., filtration or centrifugation, and the solid byproduct 4 is removed. The remaining solution 6 can be recycled to another batch run, i.e., by removing water to yield a solution comprising high boiling, water miscible solvent and acid. If necessary, additional acid 2 can be added to replenish any acid lost to degradation in the process. A reservoir of makeup solvent 9 can also be available to replace any lost or degraded high boiling solvent.

Furfural yields with sulfolane as the reaction solvent can vary depending on the specific biomass feedstock, and are typically over 80% and at as high as 99% conversion as shown in Example 1. Further, materials produced by hydrolysis and chemical conversion of C₆ sugars released from the biomass, such as levulinic acid, can be separated and sold or used in other applications. Also, certain by-products (e.g., humins) solubilized in the reaction solvent can be precipitated by addition of water or aqueous solution and then removed, thereby providing a convenient and effective way of removing these undesirable byproducts from the reaction mixture.

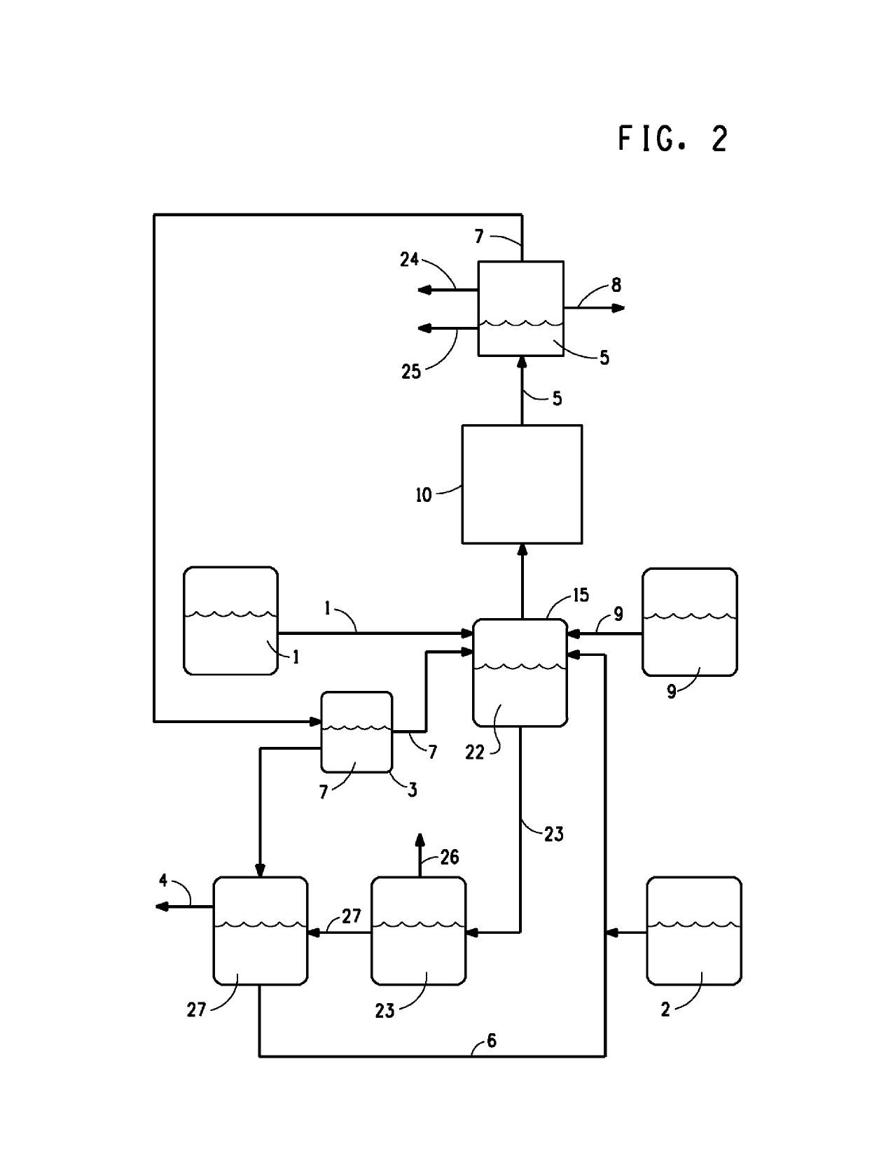

In another embodiment of the processes disclosed herein, the process is run continuously. FIG. 2 shows a schematic illustration of another exemplary reactor configuration used in the production of furfural in a continuous mode in accordance with various embodiments of the present invention. With reference to FIG. 2, the reaction vessel 15 is charged initially with a solution comprising a high boiling, water-miscible solvent and a catalytic amount of acid. The contents of the reaction vessel 15 are heated to the reaction temperature. Feedstock (biomass and/or insoluble polysaccharide) 1 is added to the hot reaction vessel 15 as dry solids, as damp solids, or as a slurry in water or solvent. Furfural is produced by acid hydrolysis and chemical conversion of the feedstock, which is then distilled from the reaction mixture 22 along with water added to reaction vessel 15, and produced by the reaction. This minimizes the residence time of furfural 8 in the acidic environment and thereby minimizes its degradation. Other useful products that may distill with furfural and water are formic acid 24 and acetic acid 25. The furfural, as well as any other desired products that may be present, are separated from the water and purified by any convenient methods known in the art, and the product furfural 8 is removed. The water 7 is either recycled to vessel 3 in the process for reuse, or is released from the process. At least a portion 23 of the reaction solution 22 is removed from reaction vessel 15 through a filter or screen (to prevent aspiration of unreacted feedstock) to another vessel. A separate distillation is carried out at pressures from 0.0001 to 0.11 MPa) to remove volatile products, that have boiling points between that of the furfural/water azeotrope and that of the high boiling, water miscible organic solvent. This allows recovery of potentially valuable byproducts such as levulinic acid 26.

The remaining contents 27 of the high boiler distillation can undergo a solids separation step to remove insoluble solids that are or suspended in 23, such as dirt, silica, insoluble salts, or any residual unconverted biomass. The recovered liquid 27 is pumped to a mixing chamber and diluted with water 7 from vessel 3. This initiates precipitation of solid byproducts 4, such as humins and lignin. The ratio of water added to the contents of the remaining liquid 27 can be from about 1:1 up to 100:1 by volume. In an embodiment, the ratio is from about 3:1 to about 20:1. The precipitated byproducts 4 are removed by any convenient means, such as filtration or centrifugation. The isolated solids 4 are washed to reclaim any reaction solvent, furfural, sugars, and other water-soluble products (e.g., levulinic acid from C₆ sugar in the biomass, acetic acid and formic acid) that were retained in the wet solids. If necessary, the washed solids are then further dried. Isolated solids 4 can be burned as an energy source. The washes can be returned to the reaction vessel 15, or are released from the process.

The precipitate-free liquid 6 separated from the solids 4 can have the pH of the solution adjusted as needed by adding makeup acid 2. This is done to replenish any acid degraded in the process. In an embodiment, the precipitate-free liquid 6 is concentrated by evaporation before mixing with an acid solution 2. The precipitate-free liquid 6 is delivered into reaction vessel 15. Any solvent 9 lost from the process, or lost by degradation can be replenished by addition to reaction vessel 15, or other streams entering 15. Thus, a continuous flow of components into and out of the reaction vessel 15 is established, distilling water, furfural, formic acid and acetic acid out of reaction vessel 15 into distillation column 10 while simultaneously distilling other volatile products such as levulinic acid 26, that have boiling points between that of the furfural/water azeotrope and that of the high boiling solvent, from at least a portion 23 coming from the reaction vessel 15, and using the feedstock solution or water 7 to precipitate and remove humins 4 from the remaining liquid 27, with the remaining liquid 6 separated from 4 flowing back to the reactor 15. The flow of liquid and solids as indicated by the arrows in FIG. 2 can occur simultaneously with the proper balance of inlet and outlet flows. For example, the flow of water 7 to the reactor 15 can be lowered during the process as water is contained in 6 which is also being brought into reactor vessel 15.

In the processes disclosed herein, the solid biomass and/or insoluble polysaccharide is substantially dissolved in a high boiling organic solvent containing an acid catalyst and furfural is produced. The furfural is stripped from the reaction mixture with water that is added to the hot solvent, which produces steam that carries the furfural away from the acidic medium. Because contact of furfural with the acidic medium is minimized, degradation of the furfural is also kept to a minimum. The biomass does not have to be pretreated or pre-sprayed with acid as in previous processes. The reaction is carried out at or near atmospheric pressure, so no exotic materials of construction are necessary. Because the biomass undergoes near complete dissolution, the residual material is flowable and easier to handle than residual solids reported for other processes. Further, certain by-products (e.g., humins and lignin) solubilized in the reaction solvent can be precipitated by addition of water or aqueous solution and then removed (e.g., by filtration), thereby providing a convenient and effective way of removing these undesirable byproducts from the reaction mixture. The higher boiling components produced in the reaction such as levulinic acid can be removed from the reaction solvent, for example, by distillation at lower pressures for further separation and purification. Also, additional useful products from the biomass such as acetic, formic and levulinic acids are produced as well, which can be separated and used or sold.

The process described above produces furfural from solid biomass and/or polysaccharide in one step at both high yield and high conversion. It is capable of operation in a batch or continuous mode. In an embodiment, the furfural yield is in the range of or 10-100%, or 20-95% or 50-92%. In another embodiment, the conversion is in the range of 50-100% or 70-100% or 90-100%. It has several advantages over known processes: Because furfural contact with the acidic medium is minimized, degradation of the furfural is also kept to a minimum. The biomass does not have to be pretreated or pre-sprayed with acid as in previous processes. The reaction can be carried out at or near atmospheric pressure without the use of high pressure.

In one embodiment of the invention disclosed herein, a process is provided comprising the steps of:
a) providing a water-miscible organic solvent and a soluble acid catalyst in a reaction vessel, wherein the boiling point of the solvent is higher than about 100° C.,
b) adding feedstock and, optionally, water in the form of liquid and/or steam to the reaction vessel to form a reaction mixture wherein
   i) the feedstock comprises solid biomass and/or insoluble polysaccharide,
   ii) the temperature of the reaction mixture is between about 100° C. and about 250° C.,
   iii) the reaction mixture pressure is between 0 MPa and about 0.21 MPa, and
   iv) the feedstock, organic solvent, and catalyst are in contact for a time sufficient to effect a reaction to produce furfural and water; and
c) removing vapors of furfural and water from the reaction mixture via reflux through a multistage distillation column;
d) condensing and collecting a solution comprising furfural and water; and
e) recovering furfural from the mixture removed in step d).

In a further embodiment of the invention disclosed herein, a process is provided comprising the steps of:
a) providing a water-miscible organic solvent and a soluble acid catalyst in a reaction vessel, wherein the boiling point of the solvent is higher than about 100° C.;
b) heating the contents of the reaction vessel to a temperature between about 100 and about 250° C.;
c) adding feedstock and, optionally, water in the form of liquid and/or steam to the reaction vessel to form a reaction mixture wherein
   i) the feedstock comprises solid biomass and/or insoluble polysaccharide,
   ii) the feedstock is present at about 1 to about 90 weight percent based on the weight of the reaction mixture,
   iii) the reaction mixture pressure is between 0 MPa and about 0.21 MPa, and
   iv) the feedstock, organic solvent, and catalyst are in contact for a time sufficient to effect a reaction to produce furfural and water;
d) removing vapors of furfural and water from the reaction mixture via reflux through a multistage distillation column;
e) condensing and collecting a solution comprising furfural and water;
f) feeding the remaining contents of the reaction vessel, or a portion thereof, through a filter or screen to a high boiler distillation vessel wherein higher boilers, including levulinic acid, are distilled at a pressure of 0 MPa to 0.21 MPa;
g) optionally, removing remaining solids from the reaction vessel for recycle into the process, or for removal from the process;
h) removing solids from the high boiler distillation vessel after the removal of high boilers in step f);
i) feeding remaining solution from the high boiler distillation vessel, or a portion thereof, to a mixing chamber and diluting the solution with water thereby precipitating water-insoluble material;
j) removing the water-insoluble material precipitated in step i); and
k) feeding the solution remaining after step j) back to the reaction vessel.

As used herein, where the indefinite article “a” or “an” is used with respect to a statement or description of the presence of a step in a process of this invention, it is to be understood, unless the statement or description explicitly provides to the contrary, that the use of such indefinite article does not limit the presence of the step in the process to one in number.

As used herein, when an amount, concentration, or other value or parameter is given as either a range, preferred range, or a list of upper preferable values and lower preferable values, this is to be understood as specifically disclosing all ranges formed from any pair of any upper range limit or preferred value and any lower range limit or preferred value, regardless of whether ranges are separately disclosed. Where a range of numerical values is recited herein, unless otherwise stated, the range is intended to include the endpoints thereof, and all integers and fractions within the range. It is not intended that the scope of the invention be limited to the specific values recited when defining a range.

As used herein, the terms “comprises,” “comprising,” “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but may include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the term “invention” or “present invention” is a non-limiting term and is not intended to refer to any single variation of the particular invention but encompasses all possible variations disclosed in the specification and recited in the claims.

As used herein, the term “about” modifying the quantity of an ingredient or reactant of the invention employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities. The term “about” may mean within 10% of the reported numerical value, preferably within 5% of the reported numerical value.

### EXAMPLES

The methods disclosed herein are illustrated in the following examples. From the above discussion and these examples, one skilled in the art can ascertain the essential characteristics of this invention, and without departing from the spirit and scope thereof, can make various changes and modifications of the invention to adapt it to various uses and conditions.

### Abbreviations

The meaning of abbreviations is as follows: “cm” means centimeter(s), “DMSO” means dimethylsulfoxide, “g” means gram(s), “h” means hour(s), “HPLC” means high pressure liquid chromatography, “min” means minute(s), “mL” means milliliter(s), “mm” means millimeter(s), “N” means normal, “PTFE” means poly(tetrafluoroethylene), “wt %” means weight percentage, “μL” means microliter(s), and “μm” means micrometer(s).

### Materials

The sources and compositions of the biomass used are presented in Table 1:

TABLE 1

| | | | | | | wt % | wt % |
|---|---|---|---|---|---|---|---|
| Bio- | | wt % | wt % | wt % | wt % | For- | Ace- |
| mass | Source | Glucan | Xylan | Arabinan | Lignin | myl | tyl |
| Corn | Best | 37.5 | 28.7 | 2.8 | 13.9 | 0.43 | 2.8 |
| cob | Cob, | | | | | | |
| | LLC, | | | | | | |
| | lowa, | | | | | | |
| | USA | | | | | | |
| Corn | USA | 33.0 | 20.1 | 2.7 | 24.1 | — | 2.8 |
| Sto- | | | | | | | |
| ver | | | | | | | |
| Ba- | Brazil, | 36.1 | 22.4 | 2.1 | 26.2 | — | 3.4 |
| gasse | South | | | | | | |
| | America | | | | | | |
| Bam- | USA | 40.8 | 21.6 | 1.0 | 24.9 | — | 3.6 |
| boo | | | | | | | |

Sulfolane was obtained from Sigma-Aldrich Corp. (St. Louis, Mo., USA).

Deionized water was used unless otherwise indicated.

### Methods

The distillate and reaction flask contents were analyzed using an HPLC instrument with a calibrated Biorad Aminex HPX-87H column. An aqueous 0.01 NH₂SO₄ isocratic mobile phase flowing at 0.6 mL/min through a column heated to 65° C. and a refractive index detector heated to 55° C. The detected amounts of xylose, furfural, formic acid, levulinic acid, and solvent were recorded.

### Example 1

### Batch Production of Furfural

The reactions were done in a 10 mL three-necked round-bottomed flask (Chemglass, Inc. Catalog No. PN CG-1507-03) containing a PTFE-coated magnetic stirring bar (VWR International, LLC Catalog No. 58949-010), a thermowell, and a threaded adapter with a cap (Chemglass, Inc. Life Sciences Catalog No. CG-350-01) and with a PTFE-lined silicone septum (National Scientific Co. Catalog No. B7995-15). The flask was connected to a vacuum-jacketed Vigreux distillation column (Chemglass, Inc. Life Sciences Catalog No. CG-1242) loaded with 8.0 g of 4 mm diameter glass beads (Chemglass, Inc. Life Sciences Catalog No. CG-1101-03). The beads were held in place with a piece of 1/16″ (1.59 mm) thick fluoropolymer that was approximately ¾″ (1.90 cm) wide by 3″ (7.62 cm) long that was either wound up into a coil or folded so that it contained pleats and was placed at the bottom of the Vigreux column to hold up the glass beads. A 20 mL plastic syringe (Beckton Dickinson & Co.) with Luer lock tip filled with water (Chemglass, Inc. Life Sciences Catalog No. PN 309661) was connected to 1/16″ (1.59 mm) diameter fluoropolymer tubing which was pierced through the septum. The syringe was controlled with a digital syringe pump. The reactions were done under a blanket of nitrogen.

Sulfolane (5 g), sulfuric acid (0.075 g) and solid biomass (0.5 g, dry weight basis) were added to the reaction flask. (Biomass loading corresponds to a (10 wt % solid biomass loading based on the weight of solvent plus biomass). The Vigreux distillation column was attached to the flask. The 1/16″ (1.59 mm) diameter fluoropolymer tube attached to the tared syringe containing water was pushed through the septum and into the reaction flask. An oil bath was heated above the desired reaction temperature. The flask was lowered into the hot oil to bring the reactor contents to the desired temperature and then the syringe pump was started. The water was added at a constant rate (typically with a set value from 0.1 to 0.4 mL/min), and the temperature of the reaction mixture was maintained as constant as possible by slight adjustments to the height of the apparatus in the oil bath. At the end of the reaction, the syringe pump was stopped, the tube was pulled from the reaction flask, and the apparatus was raised out of the oil bath.

The amount of distillate collected was weighed, internal standard (DMSO) was added to it, and the solution was then mixed until it was homogeneous (additional water was added to dilute the mixture if necessary). The reaction flask was removed from the distillation column and was weighed to determine the mass of material in the flask. Internal standard (DMSO) was added to the reaction flask and this was mixed well. The contents of the reaction flask were then transferred to a 50 mL centrifuge tube. The distillation head was washed with 3 mL of sulfolane three times and the washes were also used to wash the reactor flask each time. All the washes were combined into a 50 mL centrifuge tube which was centrifuged, and the supernatant was analyzed.

The distillate and reaction flask contents were analyzed using an HPLC instrument with a calibrated Biorad Aminex HPX-87H column. An aqueous 0.01 NH₂SO₄ isocratic mobile phase flowing at 0.6 mL/min through a column heated to 65° C. and a refractive index detector heated to 55° C. The detected amounts of xylose, furfural, formic acid, levulinic acid, and solvent were recorded. Results are presented in Table 2.

TABLE 2

| | | | | | | Selectivity | | Levulinic |
|---|---|---|---|---|---|---|---|---|
| | | | | | | to Furfural | Total FF | acid |
| | | | | | | (%) | yield, % | Yield (%) |
| | | | | | | (calculated | (calculated | (calculated |
| | | Water | | | C₅ | based | based on | based on |
| | | Addition | | Average | sugar | on | biomass C₅ | biomass |
| | | Rate | Reaction | Temp. | conversion | contained | sugar | C₆ sugar |
| Run | Biomass | (mL/min) | Time (min) | (° C.) | (%) | xylan | content) | content) |
| 1A | Corn cob | 0.1 | 40 | 168 | 86 | 71 | 61 | 27 |
| 1B | Corn cob | 0.3 | 40 | 169 | 99 | 77 | 76 | 25 |
| 1C | Corn cob | 0.4 | 40 | 169 | 99 | 85 | 84 | 30 |
| 1D | Corn cob | 0.4 | 10 | 171 | 98 | 83 | 81 | 28 |
| 1E | Corn cob | 0.4 | 25 | 168 | 99 | 82 | 82 | 26 |
| 1F | Corn cob | 0.4 | 40 | 169 | 99 | 85 | 84 | 30 |
| 1G | Corn | 0.4 | 25 | 172 | 96 | 92 | 88 | 20 |
| | stover | | | | | | | |
| 1H | Bagasse | 0.4 | 25 | 168 | 99 | 72 | 71 | 19 |
| 1I | Bamboo | 0.4 | 25 | 169 | 98 | 88 | 86 | 21 |
| 1J | Corn | 0.4 | 40 | 169 | 96 | 82 | 79 | 17 |
| | stover | | | | | | | |
| 1K | Bagasse | 0.4 | 40 | 169 | 98 | 59 | 58 | 20 |
| 1L | Bamboo | 0.4 | 40 | 168 | 98 | 84 | 83 | 27 |

### Example 2

### Continuous Production of Furfural

A 500 mL round-bottomed flask with a 29/26 ground glass joint was modified such that three threaded joints were sealed to the flask. Two of these threaded joints (Chemglass, Inc. PN CG-350-10) were used to form a compression seal with lengths of ⅛″ (0.318 cm) outer diameter fluoropolymer tubing used in the process. The third joint (Chemglass, Inc. PN CG-350-01) was sealed with a septum, and used as an extra port as needed. The modified flask was loaded with 293 g of sulfolane, and 4.5 g of sulfuric acid. The flask was connected to a vacuum jacketed distillation head with Vigreux column (Chemglass, Inc. Lifescience Company Catalog No. CG1247-10) filled with 120 g of 6 mm diameter glass beads. The distillation head was connected to a condenser that was chilled to 5° C. with a recirculating chiller. Also connected directly above the flask, was a solid addition feeder (Schenck Accurate, model 304M) arranged such that the solids would drop directly into the reaction solution, and such that a gas tight seal was attained between the inlet of the solid addition tube, and the flask. The feeder containing corncob milled to appx 1/16″ (Best Cob LLC, Iowa, 28.7 wt % xylan, 2.8 wt % arabinan and 37.5 wt % glucan, 6 wt % water). The feeder was modified with a ½ inch thick Lexan lid that was clamped and sealed to the top of the hopper compartment with foam gasket and silicon grease. The lid contained a ⅛″ threaded Swagelok adapter connected to a nitrogen source set at 0.0069 MPa. The solid addition feeder's auger was set such that it added an average of 2.2 g/min of dry corncob during operation, and a nitrogen flush maintained through the hopper and the auger, thus preventing steam from flowing into the auger during the process.

The reaction flask was lowered into a tin/bismuth metal bath heated to 260° C. in order to generate an internal reaction temperature of approximately 170° C. over the course of the reaction. One of the fluoropolymer tubes sealed in the compression joint had a glass frit with a diameter of approximately 0.75 inches attached to it to prevent aspiratiation of undissolved solids. The frit and tube were submerged in the hot reaction mixture. The other end of this tube was connected to a peristaltic pump (Masterflex PTFE pump head, PN 77390-00) which was removing the reaction mixture away from the reaction flask at approximately 0.75 mL/min; this was pumped through the tube into a small mixing chamber (Swagelok fitting, PN SS-200-3TFT) which contained a small, rotating, fluoropolymer-coated magnetic stirring bar. Deionized water was also pumped into this mixing chamber at a rate of 3 mL/min, thereby initiating precipitation of humin byproduct. A bottle of water was previously placed on a balance and was weighed at the start of the process. Over the 1.9 h reaction window for which the process was run, 352 g water was pumped through the small mixing chamber using an HPLC pump (Gilson) at a rate of approximately 3 mL/min or a 4:1 ratio relative to the incoming volume of reaction mixture. A back pressure regulator set at 0.21 MPa was installed after the HPLC pump to maintain a constant flow of water into the small mixing chamber.

After precipitation in the mixing chamber, the water/sulfolane solution containing suspended humin/lignin solid particle byproducts was transported through a switching valve and into a 102 mm stainless steel filter assembly. The 102 mm filter holder assembly contained a 1 μm polypropylene filter media (Eaton) which was used to filter the solid. The clear filtrate from the filter assembly flowed into a 200 mL screw cap glass bottle (Wilmad-LabGlass PN C-1395-250) modified by the addition of a threaded joint (Chemglass, Inc. PN CG-350-01) and a GL-45 cap (Chemglass, Inc. PN CG-1158-20) containing three ports for ⅛″ tubing. The bottle sat atop a magnetic stir plate (IKA) in order to rotate a fluoropolymer-coated magnetic stirring bar contained in the bottom of the bottle. A pH electrode (Thermo Scientific® Orion PN 911600) was inserted through and sealed with the threaded joint in this bottle. This apparatus was referred to as the pH adjustment chamber. The pH electrode was connected to a pH meter (Eutech Instruments, pH 200 Series) which controlled a micro-pump (Biochem Valve, PN 20SP1210-5TE). The pH meter adjustment level was set to 1.65. When the pH in the adjustment chamber climbed above 1.65, the pH meter controlled micro-pump added one injection of approximately 10 μL of a 10.0 wt % aqueous sulfuric acid solution into the pH adjustment chamber through a 1/16″ outer diameter fluoropolymer tube. The pH remained between 1.40-1.60, so no additional makeup acid was needed.

A ⅛″ diameter fluoropolymer tube submerged to the bottom of the pH adjustment chamber was connected to a valveless rotating reciprocating pump (pump head was from Fluid Metering Inc., PN RH00 and the drive was from Scilog, Inc.). The filtrate liquid was pulled from the pH adjustment chamber to the pump head and was then pushed through a ⅛″ diameter fluoropolymer tube into the reactor, just above the solvent level. The pump was set to deliver approximately 3.75 mL/min from the pH adjustment chamber into the reaction vessel. The total volume of the reaction mixture in the reactor was approximately 240 mL, making the reaction volume to total flow rate ratio equal to 64/min. The residence time in the flask was defined by the ratio of the volume in the reactor to the volume being recycled each minute. This residence time was 5.3 h.

Water was distilled through the reaction flask to ensure the rate of water addition to the process was equal to the rate of water from the reaction flask. When this was achieved, the corncob addition was initiated. It was added to the reaction flask in 5 min intervals at a rate of 2.2 g of dry corncob/min. After the 5 min, the addition was stopped for 10 minutes. This was followed by another 5 min addition, and stoppage for 10 min, and this method of solid addition was continued for a total time of 75 min). The average rate of cob addition during this period was 0.75 g of dry corncob/min. At the end of addition, water was added to process for an additional 35 min to complete the reaction and strip additional furfural from the reaction pot to give a total reaction time, including this final step, of 112 min.

Upon completion of the reaction, approximately 250 g of water was passed through the solids collected in the filter assembly at a rate of 10 mL/min to wash residual sulfolane and acid from the solids. The washed solids were removed from the filter assembly, and were dried in a vacuum oven yielding 2.25 g of dark solid.

The distillate (361 g) and the reaction mixture (326 g) were collected and weighed. To each was added an internal standard solution. These samples were filtered through a 0.2 micron filter and were analyzed using an HPLC instrument with a calibrated Biorad Aminex HPX-87H column. An aqueous 0.01 NH₂SO₄ isocratic mobile phase flowing at 0.6 ml/min through a column heated to 65° C. was used to analyze the sample, using a refractive index detector heated to 55° C. The detected amounts of furfural, formic acid, acetic acid, levulinic acid, and solvent were recorded. A total of 54 g of dry corncob was added to the reaction pot and a total of 7.1 g of furfural, 1.1 g of formic acid and 2.3 g of acetic acid were collected from the distillate. 3.4 g of levulinic acid was detected in the reaction mixture. The furfural molar yield was 57%, based on the C5 sugar content in the corncob.

What is claimed is:

1. A process comprising:
(d) providing a reactor comprising a distillation column disposed on top of a reaction vessel, wherein the reaction vessel contains a biomass feedstock, water, a soluble acid catalyst and a water-miscible organic solvent having boiling point of at least 100° C., wherein the biomass feedstock comprises solid biomass, insoluble polysaccharide or a mixture thereof;
(e) adding water to the reaction vessel and bringing the contents of the reaction vessel to a temperature in the range of 100-250° C. and a pressure in the range of 0.0001-0.21 MPa to form a reaction mixture for a residence time sufficient to produce a mixture of water and furfural; and
(f) removing the mixture of water and furfural from the top of the distillation column.

2. The process according to claim 1 further comprising recovering at least one of formic acid or acetic acid from the distillation column in step (c).

3. The process according to claim 1, wherein the process is a continuous process further comprising:
(d) distilling at least a portion of the contents of the reaction vessel to remove distillates including levulinic acid, that have boiling points between that of furfural/water mixture and the water-miscible organic solvent;
(e) removing solid materials from the remaining mixture of step (d) by filtration or centrifugation;
(f) adding water to the remaining mixture of step (d) and precipitating humins, lignin, and insoluble byproducts;
(g) separating the liquid from the solids produced in step (f); and
(h) adding at least one of soluble acid catalyst or water-miscible organic solvent to the solid-free liquid obtained in step (g) and adding it to the reaction vessel as in step (a).

4. The continuous process according to claim 3 further comprising:
(i) concentrating by evaporation of water at least a portion of the solid-free liquid obtained in step (g), and adding it to the reaction vessel as in step (a).

5. The continuous process according to claim 2 further comprising:
(j) separating furfural from the removed mixture of water and furfural of step (c); and
(k) using water of step (i) as the water in steps (a), (b) or (f).

6. The process according to claim 1, wherein the water-miscible organic solvent is sulfolane, polyethylene glycol, isosorbide dimethyl ether, isosorbide, propylene carbonate, poly(ethylene glycol)dimethyl ether, adipic acid, diethylene glycol, 1,3-propanediol, glycerol, gamma-butyrolactone, 2-methyl-1-pyrrolidinone, gamma-valerolactone, or mixtures thereof.

7. The process according to claim 1 wherein the acid catalyst comprises a mineral acid, a heteropolyacid, an organic acid, or a combination thereof, and wherein the acid catalyst is present in the organic solvent at 0.01-10 weight percent based on the total weight of the solution.

8. The process according to claim 1 wherein the acid catalyst is sulfuric acid or phosphoric acid.

9. The process according to claim 1 wherein the concentration of the feedstock dispersed in water is in the range of 5-90 weight percent based on the total weight of the feedstock dispersion.

10. The process according to claim 1 wherein the feedstock comprises corn cob, corn stover, sugar cane bagasse, bamboo, wood, or a combination of any of these.

11. The process according to claim 1 wherein the feedstock comprises cellulose.

12. A process comprising the steps of:
a) providing a high boiling, water-miscible organic solvent and a soluble acid catalyst in a reaction vessel, wherein the boiling point of the solvent is higher than about 100° C.,
b) adding feedstock and liquid water to the reaction vessel to form a reaction mixture wherein
i) the feedstock comprises solid biomass and/or insoluble polysaccharide,
ii) the temperature of the reaction mixture is between about 100° C. and about 250° C.,
iii) the reaction mixture pressure is between 0.0001 MPa and about 0.21 MPa, and
iv) the feedstock, organic solvent, and catalyst are in contact for a time sufficient to effect a reaction to produce furfural and water; and
d) removing vapors of furfural and water from the reaction mixture via reflux through a multistage distillation column; and
e) condensing and collecting a solution of furfural and water.
